(19) United States
(12) Patent Application Publication
(10) Pub. No.: US 2013/0331771 A1
(43) Pub. Date: Dec. 12, 2013

(54) GAS-ASSISTED DEVICE AND METHOD FOR DISPENSING BIOMATERIALS

(71) Applicant: NORDSON CORPORATION, Westlake, OH (US)

(72) Inventors: Thomas Allan Kirk, Hastings, MN (US); Todd William Sharratt, Stillwater, MN (US); Bradley D. Robb, Maple Plain, MN (US); Jon E. Hoogenakker, Inver Grove Heights, MN (US)

(73) Assignee: NORDSON CORPORATION, Westlake, OH (US)

(21) Appl. No.: 13/799,450

(22) Filed: Mar. 13, 2013

Related U.S. Application Data
(60) Provisional application No. 61/657,004, filed on Jun. 7, 2012.

(51) Int. Cl. A61M 35/00

(52) U.S. Cl. CPC A61M 35/003
USPC 604/24; 604/191

(57) ABSTRACT

A gas-assisted fluid-dispensing device configured to deliver an aerosol onto a surgical site. The fluid-dispensing device includes at least one fluid chamber configured to contain a fluid. The fluid chamber has a distal end and proximal end. A plunger extends at least partially into the fluid chamber from the proximal end. A spray nozzle tip is fluidly coupled to the distal end of the fluid chamber and is coupled to a gas supply. The tip is capable of generating aerosol with a gas from the gas supply and the fluid from the fluid chamber. A housing has an upper portion with a docking port configured to operably receive the fluid chamber. An actuating member is operably coupled to a plunger and is configured to apply a force to the plunger to discharge fluid from the fluid chamber and into the tip.

### CROSS-REFERENCE TO RELATED APPLICATIONS

This application claims the priority of U.S. Provisional Patent Application Ser. No. 61/657,004, filed on Jun. 7, 2012 (pending), the disclosure of which is incorporated by reference herein in its entirety.

The disclosures of International Patent Application No. PCT/US11/29763, filed on Mar. 24, 2011 and entitled GAS-ASSISTED FLUID DISPENSING DEVICE and U.S. application Ser. No. 61/657,008, filed Jun. 7, 2012 and entitled LOW PROFILE HANDLE PNEUMATIC ASSIST DEVICE FOR DISPENSING DUAL MATERIALS WITH OR WITHOUT A GAS ASSIST USED FOR SPRAYING MATERIALS are incorporated herein by reference in their entireties.

### FIELD OF THE INVENTION

The present invention relates generally to fluid-dispensing devices and, more particularly, to a fluid-dispensing device configured to dispense aerosols.

### BACKGROUND OF THE INVENTION

In the medical field, a surgeon routinely needs to deliver a drug or another fluid to an anatomical surface within a surgical site in a patient. Conventional manual and non-manual syringes are often used to deliver these fluids to the surgical site. For example, one known conventional syringe design includes two barrels, each containing separate fluids that are simultaneously dispensed and mixed to form a coating adapted to prevent bleeding at the surgical site. In order to spread the coating over a surface area at the surgical site, the double-barreled syringe may be coupled to a known mixing or blending spray tip, such as the FIBRIJET brand of blending tips, such as model SA-3654, that is commercially available from Micromedics of St. Paul, Minn. The blending spray tip receives the fluids from each of the two barrels, along with a pressurized gas from a pressurized gas source, to form a therapeutic aerosol that is sprayed over the surface to be coated. The therapeutic aerosol, including, for example, pain relievers, antibiotics, or coagulants, may be applied to the surgical site before, during, or after a surgical procedure.

It is widely accepted that a maximum of about 8 pounds of force should be applied to the syringes during use of such gas-assisted dispensers. However, as utility of the dispensers increases to include viscous materials and/or larger fluid containers (barrels, syringes, and so forth) the maximum force is often exceeded. This not only presents concerns for the structural integrity of the dispenser but also increases the discomfort experienced by those users having smaller hands. Thus, there exists a need for ergonomically improved dispensing devices that more efficiently dispense fluids, particularly high viscosity fluids, while increasing user comfort and reducing hand strain.

### SUMMARY

Generally, the present invention provides a gas-assisted fluid-dispensing device configured to deliver an aerosol onto a surgical site. In one illustrative embodiment the fluid-dispensing device comprises at least one fluid chamber configured to contain a fluid and including a distal end and proximal end. A plunger extends at least partially into the fluid chamber from the proximal end. A spray nozzle tip is fluidly coupled to the distal end of the fluid chamber and is configured to have a gas supply coupled thereto. The spray nozzle tip is capable of generating aerosol with a gas from the gas supply and the fluid from the fluid chamber. A housing has an upper portion with a docking port configured to operably receive the fluid chamber. An actuating member is operably coupled to the plunger and is configured to apply a force to the plunger to discharge fluid from the fluid chamber and into the spray nozzle tip. In one embodiment, first and second fluid chambers are configured to contain first and second fluids, respectively, and each of the fluid chambers includes a distal end. First and second plungers extend respectively at least partially into the first and second fluid chambers from the proximal ends thereof. The spray nozzle tip is fluidly coupled to the distal end of the first and second fluid chambers. The housing is configured with an upper portion and the docking port receives the first and second fluid chambers. The actuating member is operably coupled to the first and second plungers and is configured to apply a force to the plungers to discharge the fluids from the first and second fluid chambers and into the spray nozzle tip.

In additional aspects, the actuating member includes a toothed surface and the gas-assisted fluid-dispensing device further comprises a pawl operably configured to engage the toothed surface and to ratchet the actuating member relative to the housing. A torsion spring is provided and biases the pawl toward the toothed surface. The pawl includes a release arm configured to bias the pawl away from the toothed surface and against the bias of the torsion spring. The fluid dispensing device further comprises a holder configured to couple the first and second fluid chambers to the housing. The holder includes first and second channels configured to receive respective ones of the first and second fluid chambers. First and second keyed adaptors are coupled to respective distal ends of the first and second fluid chambers. The first channel includes a projection configured to receive the keyed adaptor of the first fluid chamber. The proximal ends of the first and second fluid chambers have an asymmetrical shape. The holder further comprises a keyed proximal end configured to receive the asymmetric proximal ends of the first and second fluid chambers when the asymmetric proximal ends are in a first orientation and to secure the asymmetric proximal end within the keyed proximal end when the asymmetric proximal ends are in a second orientation. An arm extends distally away from the holder toward the spray nozzle tip and includes a keyed surface. The spray nozzle tip includes a keyed groove configured to receive the keyed surface of the arm.

In additional aspects, the invention provides methods of operating the gas-assisted fluid-dispensing devices, such as the devices with one or more of the aspects described above.

Various additional features and advantages of the invention will become more apparent to those of ordinary skill in the art upon review of the following detailed description of the illustrative embodiments taken in conjunction with the accompanying drawings.

### BRIEF DESCRIPTION OF THE DRAWINGS

FIG. 1 is a perspective view of a gas-assisted fluid-dispensing device constructed in accordance with an illustrative embodiment of the invention.

FIG. 2 is an exploded perspective view of the device illustrated in FIG. 1.

FIG. 3A is a cross sectional view taken generally longitudinally along the device as shown in FIG. 1.

FIG. 3B is a cross sectional view similar to FIG. 3A, but illustrating actuation of a trigger associated with the device.

FIG. 3C is a cross sectional view similar to FIGS. 3A and 3B, but illustrating the actuation of a release arm associated with the device.

FIG. 4 is an enlarged perspective view of the distal end of the device.

FIG. 5 is another exploded perspective view illustrating the device of FIG. 1.

### DETAILED DESCRIPTION

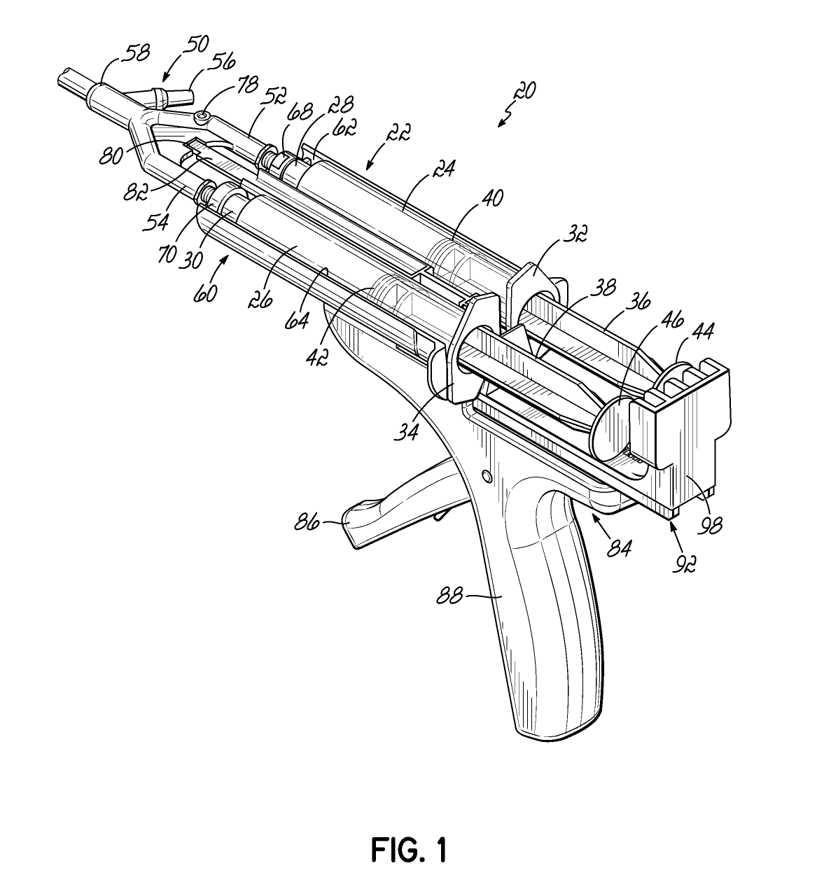

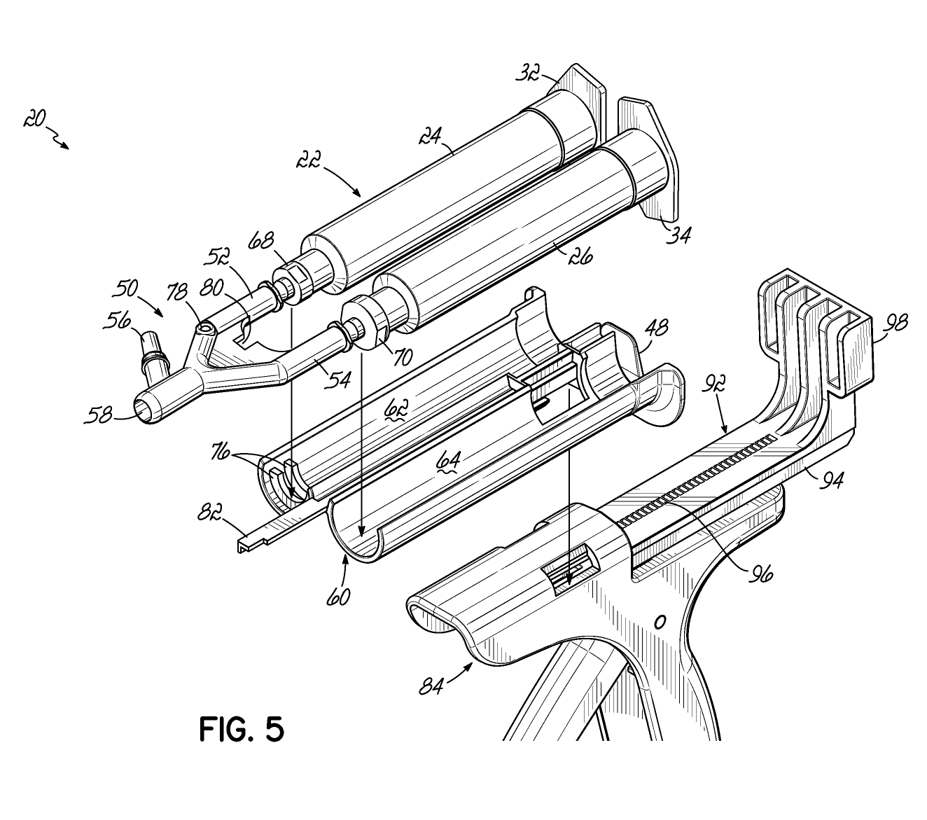

Turning now to the figures, and particularly FIGS. 1, 2, 4 and 5, one embodiment of a gas-assisted fluid dispenser 20 is shown and described in detail. The dispenser 20 includes a fluid-dispensing device 22 having a least one fluid source. The fluid source may include, for example, a dual-syringe having one or more parallel fluid chambers (two fluid chambers 24, 26 are shown) for simultaneously dispensing one or more fluids, such as topical or therapeutic medicinal agents. Moreover, it would be readily appreciated that if more than one fluid is administered, equal volume need not be dispensed. Instead, the volume of a first fluid dispensed from the first fluid chamber 24 may be larger than the volume of a second fluid dispensed from the second fluid chamber 26. Each of the fluid chambers 24, 26 includes a tapered distal end 28, 30, a winged proximal end 32, 34, and a cylinder (not shown) extending therebetween. A plunger 36, 38 resides, at least partially, within the cylinder of each fluid chamber 24, 26 and extends proximally therefrom. Each plunger 36, 38 may be constructed in a known, conventional manner and include a distally-positioned stopper 40, 42 and a proximally-positioned plunger head 44, 46.

A spray nozzle tip 50 is coupled to the fluid-dispensing device 22. And, because the dual-syringe of the illustrative embodiment includes two fluid chambers 24, 26, the spray nozzle tip 50 is illustrated as a Y-connector attached to both fluid chambers 24, 26. The spray nozzle tip 50 further includes a gas inlet 56 configured to receive a gas line (not shown). Thus, the spray nozzle tip 50, as shown, includes two fluid inlets 52, 54, one gas inlet 56, and one outlet 58. One of ordinary skill in the art would readily appreciate that the shape of the outlet 58 may be configured to provide a desired aerosol effect. That is, a desired direction and/or spread of the resultant aerosol, along with the gas pressure, may be determined by incorporating a particular design for the outlet 58.

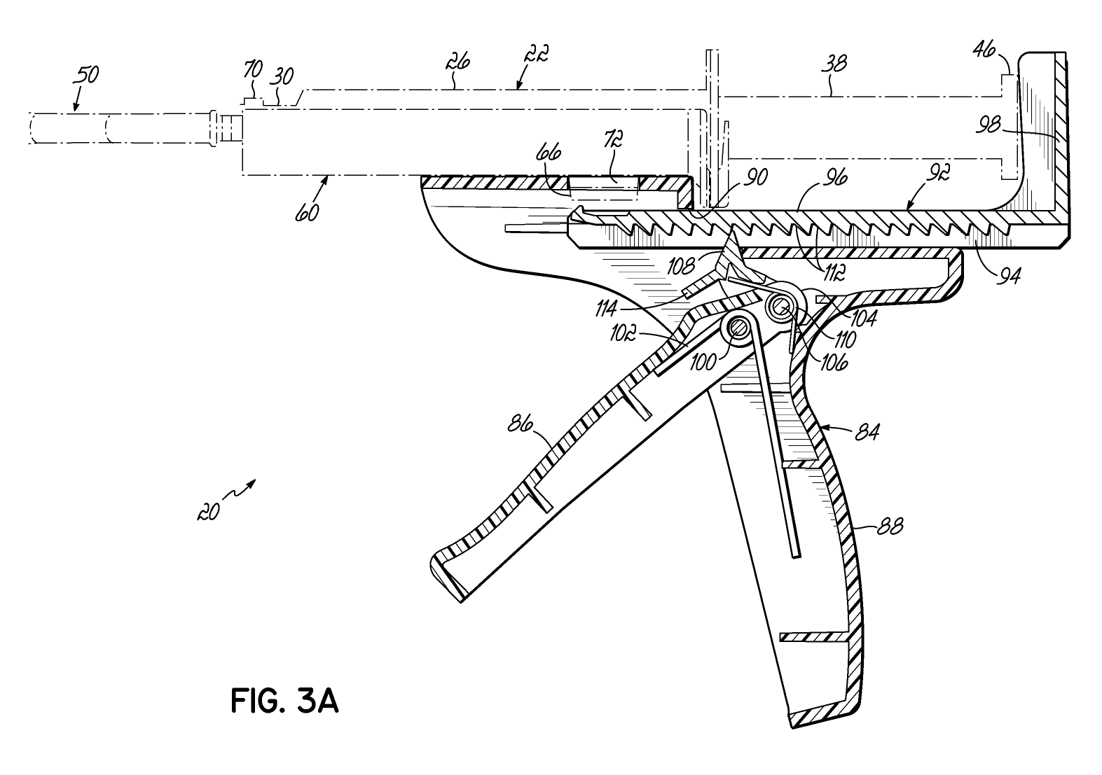

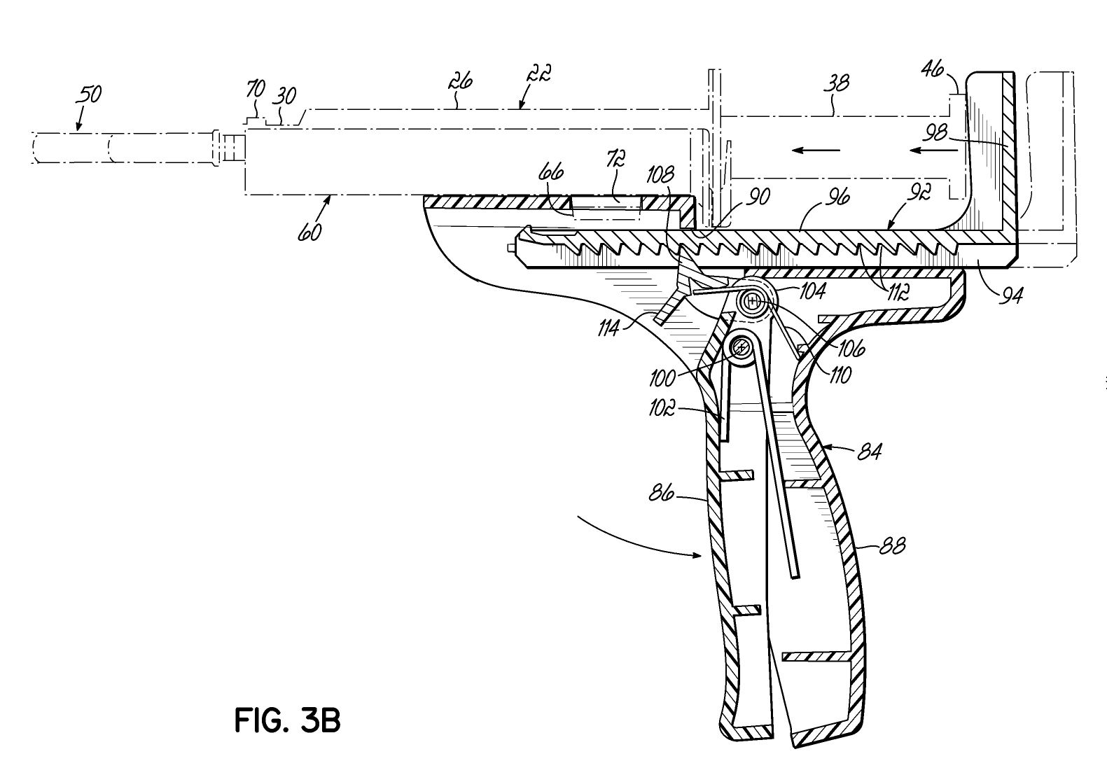

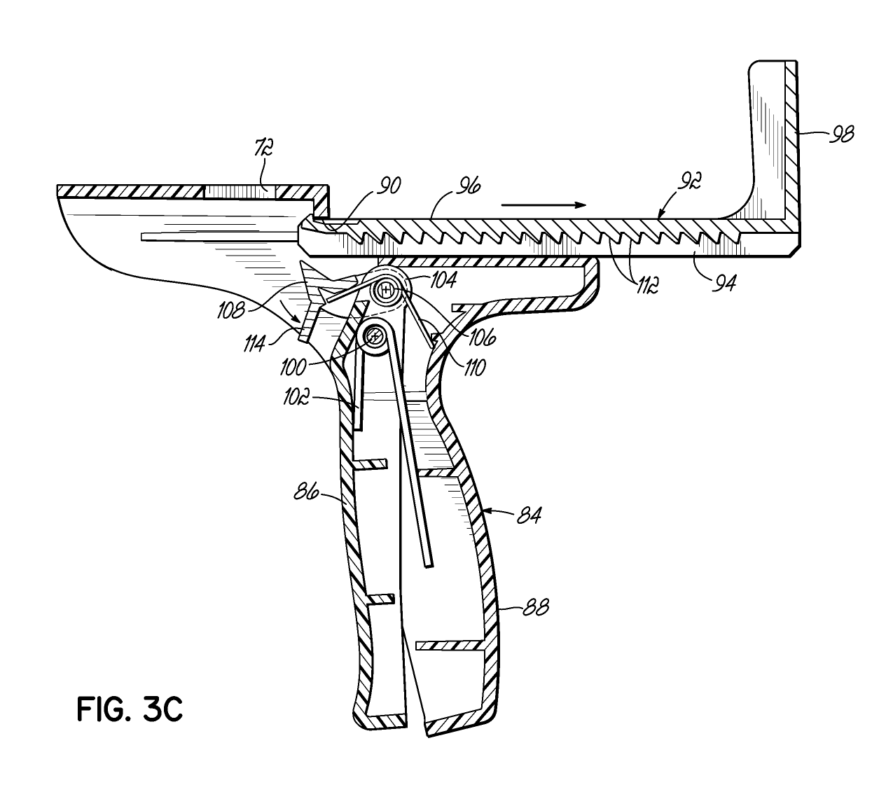

The fluid-dispensing device 22 is supported within a source holder 60, which may have a molded polymeric material construction that is sized and shaped to accommodate the fluid chambers 24, 26. Accordingly, various sizes, shapes, and configurations of holders 60 are possible for supporting one or more fluid chambers 24, 26 of similar or varying sizes. In the illustrative embodiment, the holder 60 includes first and second channels 62, 64 that are shaped and sized to receive the two fluid chambers 24, 26. The holder 60 further includes one or more tabs 66 (FIGS. 3A and 3B), which are configured to couple the holder 60 to a docking port 72 (FIG. 3C) of the dispenser 20. The docking port 72 may include one or more slots that receive respective ones of the one or more tabs 66 (FIGS. 3A and 3B).

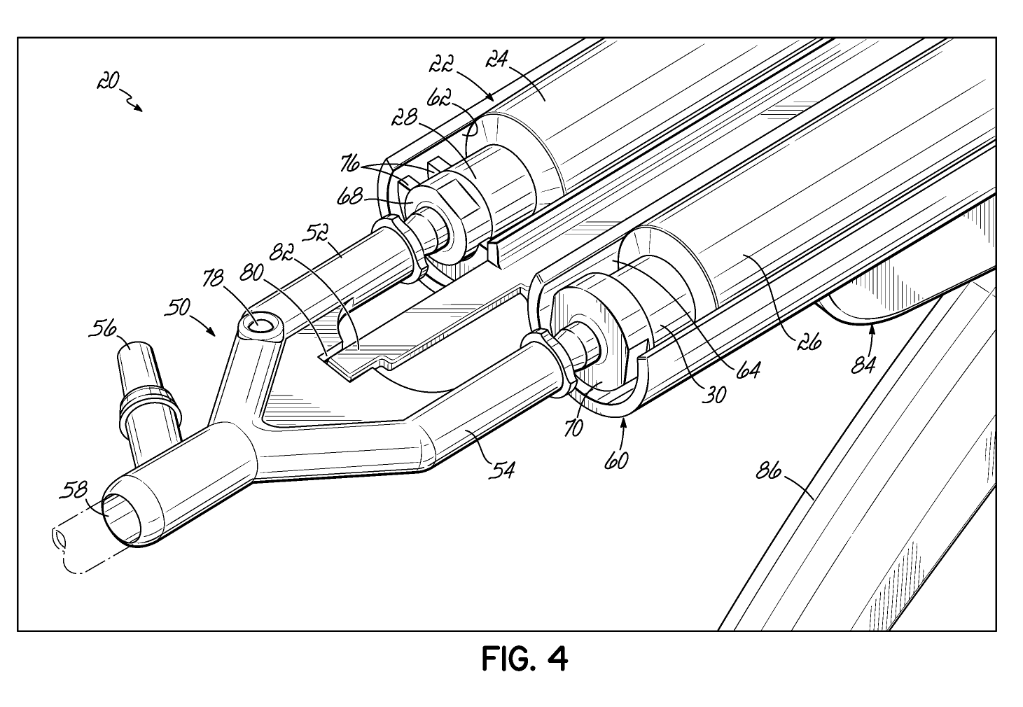

To facilitate consistent assembly of the fluid-dispensing device 22 with the holder 60, the first fluid chamber 24 containing a first fluid is loaded into the first channel 62 of the holder 60 while the second fluid chamber 26 containing a second fluid is loaded into the second channel 64 of the holder 60. The distal tapered ends 28, 30 of the fluid chambers 24, 26 each may include one of two keyed adaptors 68, 70, embodiments of which are shown in FIG. 4. The first and second keyed adaptors 68, 70 may vary in at least one structural dimension, which, as shown, are different diameters. Each adaptor 68, 70 further includes a distally-extending port, such as a luer, configured to be coupled to the inlets 52, 54 of the nozzle tip 50.

In addition to the keyed adaptors 68, 70, the distal ends of the first and second channels 62, 64 of the holder 60 may be particularly configured to receive one of the two keyed adaptors 68, 70. More specifically and in accordance with the exemplary embodiment, the first channel 62 includes a structured surface, such a projection, which is illustrated herein as one or more ribs 76 that reduce the inner diameter of the first channel 62 to approximately the outer diameter of the first keyed adaptor 68. Meanwhile, the second channel 64, remaining devoid of diameter-reducing ribs, remains sufficiently large so as to receive the larger outer diameter of the second keyed adaptor 70. As a result, and when the fluid chambers 24, 26 are loaded into the holder 60, the first fluid within the first fluid chamber 24 is necessarily loaded into the first channel 62 of the holder 60 having the ribs 76 therein to support the smaller outer diameter adaptor 68. The second chamber 26 containing the second fluid is then necessarily loaded into the second channel 64.

If desired, and as shown, the winged proximal ends 32, 34 of the fluid chambers 24, 26 may be asymmetrically shaped with respect to a keyed proximal end 48 of the holder 60. As such, the winged proximal ends 32, 34 in a first orientation may be directed into the proximal end 48 of the holder 60 and then rotated to a second orientation with respect to the proximal end 48, which secures the winged proximal ends 32, 34 thereto.

With the fluid chambers 24, 26 coupled to the holder 60, the nozzle tip 50 is coupled to the luers of the keyed adaptors 68, 70 such that the first inlet 52 is coupled to the first keyed adaptor 68 and the second inlet 54 is coupled to the second keyed adaptor 70.

In some embodiments, the nozzle tip 50 may further include an indicia 78, such as the imprinted donut of the first arm of the Y-connector, which would be understood to reside on the same side during each use of the nozzle tip 50. However, use is not prevented by such indicia 78 and a user that is quickly assembling the dispenser 20 or otherwise unaware of the laboratory or surgical suite's standard operating procedures may inadvertently rotate the nozzle tip 50 such that the indicia 78 is flipped to the opposing side during use. While this switch may be seemingly benign, in those embodiments where the first fluid reacts with the second fluid to form a coagulant or other extremely viscous, gelatinous, or adhesive material, this simple reversal of the Y-connector with respect to the first and second fluids may result in a chemical reaction within the channels of the Y-connector with residual first and second fluids therein. With sufficient residual material and/or reactivity, the channels of the Y-connector may become clogged, rendering the nozzle tip inoperable.

To prevent such reversal of the nozzle tip 50, the Y-connector may further be keyed with a groove 80 so as to receive an arm 82 protruding distally from the holder 60, which prevents reversal of the nozzle tip 50 from its accepted arrangement (with the indicia 58 proper positioned) with respect to the fluid chambers 24, 26.

A housing 84 of the dispense 20 may be constructed as two halves from a moldable polymeric material, which may be joined by screws to enclose an internal cavity. A trigger 86 is coupled to the housing 84 and operably coupled to the fluid-dispensing device 22. The housing 84 further includes a first, upper housing portion and a second, lower housing portion that is configured as a handle 88 extending angularly downward from the upper housing portion. The upper housing portion includes a slot 90 through which an actuating member 92 for the fluid-dispensing device 22 extends. That is, the actuating member 92 is operably coupled to the fluid chambers 24, 26 for dispensing the fluids therefrom and into the spray nozzle tip 50. As shown, the actuating member 92 is a T-shaped slide having an elongated base portion 94 that extends through the slot 90 and configured to slide horizontally relative thereto. A top surface of the base portion 94 includes a longitudinal array of indentations 96 extending a substantial portion of the length of the base portion. A spring positioned within the cavity between the top surface of the base portion and an inner surface of the upper housing portion engages one of the indentations 96 and is configured to maintain the relative position of the actuating member 92 during operation and reloading of the fluid-dispensing device 22. Additional details of the operation of the spring may be found in International Patent Application No. PCT/US11/29763.

The actuating member 92 further includes an upright portion 98 that extends substantially orthogonally upward from the base portion 94 and is configured to engage the plunger heads 44, 46 of the plungers 36, 38 and actuate the plungers 36, 38 to dispense the fluid to the spray nozzle tip 50.

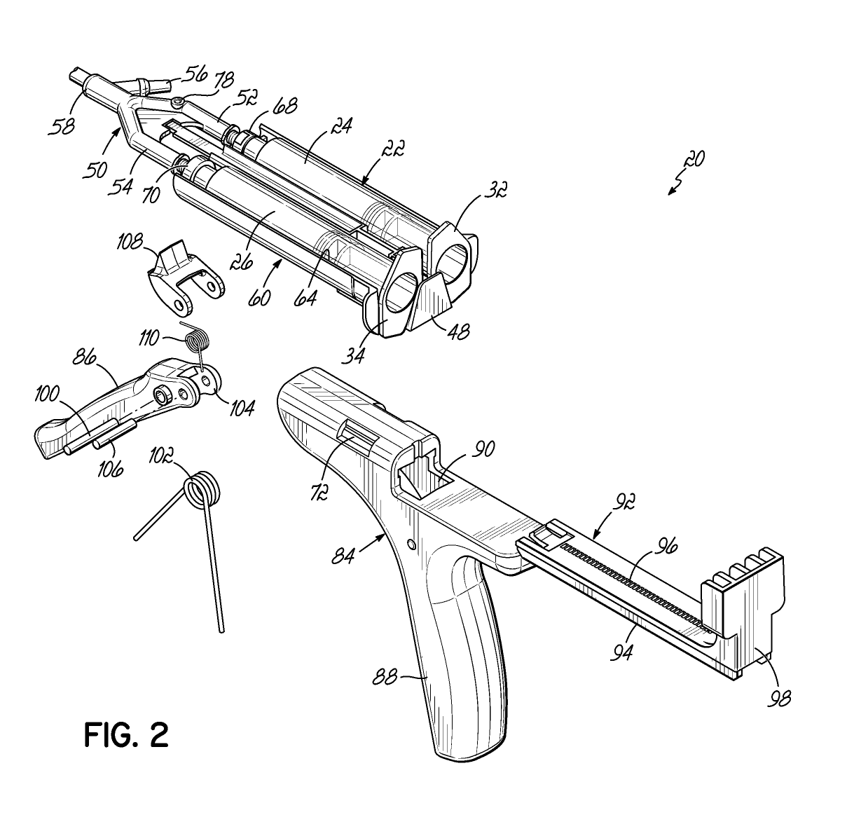

With reference now to FIGS. 2 and 3A-3C, one method of actuating the trigger 86 to dispense fluid from the fluid chambers 24, 26 into spray nozzle tip 50 is shown. The trigger 86 is operably coupled to the housing 84 at a first pivot point, represented by a trigger pin 100, and extends distally therefrom. The length of the trigger 86 may be selected to create a mechanical advantage over direct manipulation of the plungers 36, 38, for example, a 4:1 mechanical advantage. A first torsion spring 102 operably coupled to the trigger 86 at the first pivot point 100 is configured to bias the trigger 86 away from the handle 88.

A proximal end 104 of the trigger 86 is positioned within housing 84 and includes a pawl pin 106 defining a second pivot point for operating a pawl 108 and a second torsion spring 110. The pawl 108 extends angularly upwardly from the pawl pin 106 and engages a linear array of teeth 112 formed in a bottom surface of the base portion 94 of the actuating member 92. The second torsion spring 110 biases the pawl 108 upwardly and away from the trigger 86 toward the plurality of teeth 112 and with respect to the bottom portion of the housing 84.

In use, and by engaging the trigger 86, i.e., directing the trigger 86 toward the handle 88, the proximal end 104 of the trigger 86 moves distally within the cavity (e.g., toward the spray nozzle tip 50). Because the pawl 108 is coupled to the proximal end 104 of the trigger 86 at the pawl pin 106, movement of the trigger 86 also causes the pawl 108 to rotate toward the distal direction while the second torsion spring 110 continues to bias the pawl 108 upwardly. The rotational movement of the pawl 108 with continued upward bias creates a forwardly-directed force onto a rear surface of the tooth 112 engaged by the pawl 108 and will direct the slide forward. With sufficient engagement of the trigger 86 and forward movement of the pawl 108, the actuating member 92 moves forward a sufficient distance so that the pawl 108, when the trigger 86 is released, ratchets along the linear array of teeth 112 to engage a tooth 112 positioned proximate the now rest position.

This ratcheting movement of the actuating member 92 causes the upright portion 98 of the actuating member 92 to actuate the plungers 36, 38. Each engagement (or squeezing) of the trigger 86 is thus converted into a horizontal, distally-directed translation of the upright portion 98 and thereby directs the plungers 36, 38 into the cylinders of the fluid chambers 24, 26. As the plungers 36, 38 move inwardly, the fluid is compressed within each of the respective fluid chambers 24, 26 and a volume of the fluid that is proportional to the linear displacement of the plungers 36, 38 is dispensed from the tapered distal ends into the spray nozzle tip 50. Accordingly, metering of the amount of fluid dispensed may be accomplished by the number of trigger compressions, e.g., each complete trigger engagement may cause the pawl 108 to engage every tooth 112 or, alternatively, engage only selected teeth 112.

Before, during, and after each trigger squeeze, the dispensing gas is directed into the gas inlet 56 of the nozzle tip 50 from a regulator (not shown) having a foot pedal (not shown) control. In this way, the gas flow may be initiated prior to the release fluids from the fluid chambers 24, 26 into the spray nozzle tip 50 such that when the fluids are released, the fluids mixed with the flowing gas, atomizes or disperses, as a treatment aerosol from the outlet of the spray nozzle tip 50. The surgeon may continue dispensing the treatment aerosol by further compressing the trigger 86. Without further compression of the trigger 86, only the dispensing gas is released from the spray nozzle tip 50. This further dispensing of gas helps in clearing the passages of the nozzle tip 50 in preparation for future use.

After some use, it may be necessary to reload the dispenser 20 with another fluid dispensing device 22 for use with the same patient or in preparation for another surgery. Accordingly, the fluid-dispensing device 22 may need to be removed and replaced with another fluid-dispensing device 22. Because the pawl 108 is designed as a one-way ratchet, reverse movement of the actuating member 92 is not possible without first releasing the pawl 108 from the plurality of teeth 112. A release arm 114, which is coupled with the pawl 108, is accessible to the surgeon such that rearward biasing of the release arm 114 (i.e., toward the trigger 86), with or without a trigger pull, rotates the pawl 108 against the bias of the second torsion spring 110 and rotates the pawl 108 about the pawl pin 106 to withdraw the pawl 108 from the linear array of teeth 112 (FIG. 3C). Accordingly, the actuating member 92 may be retracted rearwardly and the fluid dispensing device 22 with holder 60 removed.

In accordance with the illustrated configuration, described in some detail herein, the overall movement of the trigger 86 is minimized and reduces the overall operational profile of the dispenser 20 such that users having smaller hands may more easily squeeze the trigger 86 and operate the dispenser 20 as described herein.

While the invention has been illustrated by the description of embodiments thereof, and while the embodiments have been described in considerable detail, it is not intended to restrict or in any way limit the scope of the appended claims to such detail. Additional advantages and modifications will readily appear to those skilled in the art. Therefore, the invention in its broadest aspects is not limited to the specific details shown and described. The various features disclosed herein may be used in any combination necessary or desired for a particular application. Consequently, departures may be made from the details described herein without departing from the spirit and scope of the claims which follow.

What is claimed is:

1. A gas-assisted fluid-dispensing device configured to deliver an aerosol onto a surgical site, the fluid-dispensing device comprising:
first and second fluid chambers configured to contain first and second fluids respectively, the first and second fluid chambers each including a distal end and a proximal end;
first and second plungers extending respectively at least partially into the first and second fluid chambers from the proximal ends thereof;
a spray nozzle tip fluidly coupled to the distal ends of the first and second fluid chambers and configured to have a gas supply coupled thereto, the spray nozzle tip capable of generating the aerosol with a gas from the gas supply and the fluids from the first and second fluid chambers;
a housing having a handle and an upper portion having a docking port configured to operably receive the first and second fluid chambers; and
an actuating member operably coupled to the first and second plungers and configured to apply a force to the first and second plungers to discharge the fluids from the first and second fluid chambers and into the spray nozzle tip.

2. The gas-assisted fluid-dispensing device of claim 1, wherein the actuating member includes a toothed surface, the gas-assisted fluid dispensing device further comprising:
a pawl operably configured to engage the toothed surface and to ratchet the actuating member relative to the housing.

3. The gas-assisted fluid-dispensing device of claim 2, further comprising:
a torsion spring configured to bias the pawl toward the toothed surface.

4. The gas-assisted fluid-dispensing device of claim 3, wherein the pawl includes a release arm configured to bias the pawl away from the toothed surface and against the bias of the torsion spring.

5. The gas-assisted fluid-dispensing device of claim 1, further comprising:
a holder configured to couple the first and second fluid chambers to the housing, the holder having first and second channels configured to receive respective ones of the first and second fluid chambers; and
first and second keyed adaptors coupled to respective distal ends of the first and second fluid chambers,
wherein the first channel includes a projection configured to receive the keyed adaptor of the first fluid chamber.

6. The gas-assisted fluid-dispensing device of claim 5, wherein the proximal ends of the first and second fluid chambers have an asymmetrical shape, the holder further comprising:
a keyed proximal end configured to receive the asymmetric proximal ends of the first and second fluid chambers when the asymmetric proximal ends are in a first orientation and to secure the asymmetric proximal ends within the keyed proximal end when the asymmetric proximal ends are in a second orientation.

7. The gas-assisted fluid-dispensing device of claim 1, further comprising:
a holder configured to couple the first and second fluid chambers to the housing; and
an arm extending distally away from the holder toward the spray nozzle tip, the arm having a keyed surface,
wherein the spray nozzle tip includes a keyed groove configured to receive the keyed surface of the arm.

8. A gas-assisted fluid-dispensing device configured to deliver an aerosol onto a surgical site, the fluid-dispensing device comprising:
a fluid chamber configured to contain a fluid, the fluid chamber including a distal end and a proximal end;
a plunger extending at least partially into the fluid chamber from the proximal end;
a spray nozzle tip fluidly coupled to the distal end of the fluid chamber and configured to have a gas supply coupled thereto, the spray nozzle tip capable of generating the aerosol with a gas from the gas supply and the fluid from the fluid chamber;
a housing having an upper portion with a docking port configured to operably receive the fluid chamber; and
an actuating member operably coupled to the plunger and configured to apply a force to the plunger to discharge fluid from the fluid chamber and into the spray nozzle tip.

9. The gas-assisted fluid-dispensing device of claim 8, wherein the actuating member includes a toothed surface, the gas-assisted fluid dispensing device further comprising:
a pawl configured to engage the toothed surface and to ratchet the actuating member relative to the housing.

10. The gas-assisted fluid-dispensing device of claim 9, further comprising:
a torsion spring configured to bias the pawl toward the toothed surface.

11. The gas-assisted fluid-dispensing device of claim 10, wherein the pawl includes a release arm configured to bias the pawl away from the toothed surface and against the bias of the torsion spring.

12. The gas-assisted fluid-dispensing device of claim 8, further comprising:
a holder configured to couple the fluid chamber to the housing, the holder having a channels configured to receive the fluid chamber.

13. The gas-assisted fluid-dispensing device of claim 12, wherein the proximal end of the fluid chamber has an asymmetrical shape, the holder further comprising:
a keyed proximal end configured to receive the asymmetric proximal end of the fluid chamber when the asymmetric proximal end is in a first orientation and to secure the asymmetric proximal end within the keyed proximal end when the asymmetric proximal end is in a second orientation.

14. The gas-assisted fluid-dispensing device of claim 8, further comprising:
a holder configured to couple the fluid chamber to the housing; and
an arm extending distally away from the holder toward the spray nozzle tip, the arm having a keyed surface,
wherein the spray nozzle tip includes a keyed groove configured to receive the keyed surface of the arm.

15. A gas-assisted fluid-dispensing device configured to deliver an aerosol onto a surgical site, the fluid-dispensing device comprising:
first and second fluid chambers configured to contain first and second fluids respectively, the first and second fluid chambers each including a distal end and a proximal end
first and second plungers extending respectively at least partially into the first and second chambers from the proximal ends thereof;
a spray nozzle tip fluidly coupled to the distal ends of the first and second fluid chambers and configured to have a gas supply coupled thereto, the spray nozzle tip capable of generating the aerosol with a gas from the gas supply and the fluids from the first and second fluid chambers;
a housing having an upper portion with a docking port configured to operably receive the first and second fluid chambers;
an actuating member operably coupled to the first and second plungers and configured to apply a force to the plungers to discharge the fluids from the first and second fluid chambers and into the spray nozzle tip, the actuating member including a tooth surface thereon;
a pawl configured to engage the toothed surface and to ratchet the actuating member relative to the housing; and
a torsion spring configured to bias the pawl toward the toothed surface.

16. The gas-assisted fluid-dispensing device of claim 15, wherein the pawl includes a release arm configured to bias the pawl away from the toothed surface and against the bias of the torsion spring.

17. A method of operating the gas-assisted fluid-dispensing device of claim 1, the method comprising:
actuating the gas supply so as to supply a gas to the spray nozzle tip;
moving the actuating member to cause delivery of the fluid from the first and second fluid chambers into the spray nozzle tip;
atomizing the fluid within the spray nozzle tip with the pressurized gas to form the aerosol; and
dispensing the aerosol from the spray nozzle tip onto the surgical site.

18. A method of operating the gas-assisted fluid-dispensing device of claim 9, the method comprising:
actuating the gas supply so as to supply a gas to the spray nozzle tip;
moving the actuating member to cause delivery of the fluid from the fluid chamber into the spray nozzle tip;
atomizing the fluid within the spray nozzle tip with the pressurized gas to form the aerosol; and
dispensing the aerosol from the spray nozzle tip onto the surgical site.
